(19) United States
(12) Patent Application Publication
(10) Pub. No.: US 2024/0156639 A1
(43) Pub. Date: May 16, 2024

(54) CONTACT LENSES WITH INTEGRATED ELECTRONICS AND METHOD OF MAKING THE SAME

(71) Applicant: Technology Innovation Institute — Sole Proprietorship LLC, Masdar City (AE)

(72) Inventors: Myriam Ghodhbane, Abu Dhabi (AE); Chiara Busa, Abu Dhabi (AE); Juveiriah M. Ashraf, Abu Dhabi (AE); Sunil Lonkar, Abu Dhabi (AE); Zainab Karam, Abu Dhabi (AE)

(73) Assignee: Technology Innovation Institute — Sole Proprietorship LLC, Masdar City (AE)

(21) Appl. No.: 18/508,806

(22) Filed: Nov. 14, 2023

Related U.S. Application Data
(60) Provisional application No. 63/383,785, filed on Nov. 15, 2022.

(51) Int. Cl. A61F 9/00; B29D 11/00; G02C 7/04

(52) U.S. Cl. CPC A61F 9/0017; B29D 11/00038; B29D 11/00096; G02C 7/049; B29K 2105/0061

(57) ABSTRACT

A contact lens may include a lens comprising a hydrogel configured to be worn on an eyeball. A contact lens may include an electronic component fully encapsuled within the lens, wherein the electronic component may include a sensor, a battery, a supercapacitor, an electronically controlled microfluidic channel, or combinations thereof. Methods for producing such a contact lens are also disclosed.

### CROSS-REFERENCE TO RELATED APPLICATIONS

This application claims priority to U.S. Provisional Patent Application No. 63/383,785, which was filed on Nov. 15, 2022, the entire contents of which are incorporated by reference herein.

### FIELD

The present disclosure relates generally to contact lenses with integrated electronic components and methods of making the same. More specifically, this disclosure relates to smart contact lenses with ensured biocompatibility and wide applicability which may be produced using scalable and fast methods.

### BACKGROUND

Flexible electronics have seen much advancement in recent years, with technologies developed to address numerous health and wellness needs in the form of portable, implantable, and wearable devices. While electronics have improved markedly in the past decade, methods of producing ever-smaller devices in scalable and affordable ways continue to drive development of new technologies.

“Smart” contact lenses which have electronics integrated into the lens and can be worn in the eye have a wide range of potential uses, including traditional vision correction, health monitoring, drug delivery, and digital entertainment. However, integrating electronics into contact lenses also presents challenges. Preventing mechanical failure and leakage of hazardous components is extremely important to maintaining high safety standards, and the size and thickness of the lens must be carefully controlled to ensure comfort for the user.

Smart contact lenses are commonly produced by either printing the electronics onto the surface of the lens, or by integrating the electronic components between two lenses. The first method leads to biocompatibility issues, since the electronic components are exposed and may be harmful to the eye of the user. The second method results in a lens with excessive thickness that may lead to discomfort. There remains a need for smart contact lenses with the required biocompatibility and thickness, particularly lenses that can be produced via high-throughput and economically viable methods.

### SUMMARY

There is provided a contact lens, which includes a lens including a hydrogel configured to be worn on an eyeball, and an electronic component fully encapsulated within the lens.

In embodiments, the hydrogel includes polyvinyl alcohol, (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl]propyl methacrylate, silicone, polyethylene glycol, or combinations thereof.

In embodiments, the electronic component according to any of the above embodiments includes at least one of a sensor, a battery, a supercapacitor, an electronically controlled microfluidic channel, an antenna, a near-field communication chip, a micro-near-field communication chip, or combinations thereof. In embodiments, the electronic component according to any of the above embodiments includes circuital bio-compatible components. In embodiments the electronic component includes carbon nanotubes, carbon allotropes, cellulose, or combinations thereof.

In embodiments, the contact lens according to any of the above embodiments has a thickness of about 0.05 mm to about 0.5 mm. In embodiments, the contact lens according to any of the above embodiments has a thickness of about 0.05 mm to about 0.3 mm. In embodiments, the contact lens is reusable.

In embodiments, the contact lens according to any of the above embodiments further includes a pharmaceutical which has been impregnated into the lens. In embodiments, the pharmaceutical includes an antihistamine, an ophthalmic drug, or combinations thereof. In embodiments, the contact lens is used in combination with a monitoring system to administer the pharmaceutical at set time intervals or as needed.

There is provided a method of producing a contact lens, including steps of heating a solvent to a temperature of about 50° C. to about 100° C., combining the solvent with a hydrogel precursor at a temperature of about 25° C. to about 100° C. to form a hydrogel, placing an amount of the hydrogel into a mold containing at least one well which contains an electronic component, and setting the hydrogel to form a contact lens with the electronic component fully encapsulated therein.

In embodiments, the solvent includes water, dimethylsulfoxide, chloroform, dichloromethane, methanol, ethanol, or combinations thereof. In embodiments, the solvent according to any of the above embodiments includes water and dimethylsulfoxide in a ratio of about 50:50 to about 10:90.

In embodiments, the hydrogel precursor according to any of the above embodiments includes polyvinyl alcohol, (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl]propyl methacrylate, silicone, polyethylene glycol, or combinations thereof. In embodiments, combining the solvent with the hydrogel precursor according to any of the above embodiments includes stirring, sonicating, or combinations thereof.

In embodiments, the amount of hydrogel according to any of the above embodiments placed into the mold is about 0.1 mL to about 0.5 mL per contact lens. In embodiments, the electronic component according to any of the above embodiments includes at least one of a sensor, a battery, a supercapacitor, an electronically controlled microfluidic channel, or combinations thereof. In embodiments, the mold according to any of the above embodiments includes a plurality of wells containing an electronic component, such that a plurality of contact lenses with the electronic component fully encapsulated therein are formed simultaneously. In embodiments, setting the hydrogel according to any of the above embodiments includes physical crosslinking at a temperature of about −90° C. to about 25° C. for a time of about 4 hours to about 24 hours.

In embodiments, the method according to any of the above embodiments further includes impregnating the contact lens with a pharmaceutical such that the contact lens may be used for drug delivery. In embodiments, impregnating the contact lens with the pharmaceutical according to any of the above embodiments includes soaking the contact lens in a solution comprising the pharmaceutical, adding the pharmaceutical to the hydrogel, incorporating an electronically controlled microfluidic channel comprising the pharmaceutical into the contact lens, or combinations thereof.

### DRAWINGS

Aspects, features, benefits, and advantages of the embodiments described herein will be apparent with regard to the following description, appended claims, and accompanying drawings where:

FIG. 1 is an illustrative diagram of a contact lens, according to embodiments of the present disclosure.

FIG. 2 is a flow chart of a method of making a contact lens, according to embodiments of the present disclosure.

FIGS. 3A-3C are graphs of transparency of hydrogels formed with 5 wt. % PVA, over a time of 8 hours to 96 hours. FIG. 3A depicts the transparency of hydrogels containing 5 wt. % PVA at a freezing temperature of −12° C.; FIG. 3B depicts the transparency of hydrogels containing 5 wt. % PVA at a freezing temperature of −85° C.; and FIG. 3C compares the transparency of hydrogels containing 5 wt. % PVA at a freezing temperature of −85° C. and −12° C. over 24 hours.

FIGS. 4A-4C are graphs of transparency of hydrogels formed with 7 wt. % PVA, over a time of 8 hours to 96 hours. FIG. 4A depicts the transparency of hydrogels containing 7 wt. % PVA at a freezing temperature of −12° C.; FIG. 4B depicts the transparency of hydrogels containing 7 wt. % PVA at a freezing temperature of −85° C.; and FIG. 4C compares the transparency of hydrogels containing 7 wt. % PVA at a freezing temperature of −85° C. and −12° C. over 24 hours.

FIGS. 5A-5C are graphs of transparency of hydrogels formed with 10 wt. % PVA, over a time of 8 hours to 96 hours. FIG. 5A depicts the transparency of hydrogels containing 10 wt. % PVA at a freezing temperature of −12° C.; FIG. 5B depicts the transparency of hydrogels containing 10 wt. % PVA at a freezing temperature of −85° C.; and FIG. 5C compares the transparency of hydrogels containing 10 wt. % PVA at a freezing temperature of −85° C. and −12° C. over 24 hours.

FIG. 6 is a graph of water content as a function of crosslinking time for hydrogels containing 5 wt. %, 7 wt. %, and 10 wt. % PVA, according to embodiments of the present disclosure.

FIG. 7 is a graph of oxygen permeability as a function of crosslinking time for hydrogels containing 5 wt. %, 7 wt. %, and 10 wt. % PVA, according to embodiments of the present disclosure.

FIG. 8 is a graph of percentage of swelling over time for lenses containing 10 wt. % PVA, according to embodiments of the present disclosure.

FIG. 9A is a cyclic voltammogram evaluating the potential window of the hydrogels according to embodiments of the present disclosure. FIG. 9B is a cyclic voltammogram of the hydrogels containing 10 wt. % PVA evaluating different scan rates.

FIG. 10A and FIG. 10B show scanning electron microscopy of the contact lenses which were prepared according to an embodiment of the present disclosure.

### DETAILED DESCRIPTION

According to embodiments of the present disclosure, a contact lens to be worn on an eyeball with integrated electronic components fully encapsulated therein is provided. The contact lens may be in the form of a thin circular membrane with a convex shape and may include a hydrogel and an electronic component that is fully encapsuled by the hydrogel, such that no part of the electronic component is in contact with the eyeball. The contact lens may be used for traditional vision correction, drug delivery, monitoring of health parameters, or other uses. The electronic component may include batteries, supercapacitors, sensors, electronically controlled microfluidic channels, or other electronics capable of being integrated into a contact lens. The present disclosure provides a contact lens that offers safety and biocompatibility, and further ensures comfort for the user by maintaining a thin profile throughout the contact lens.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, the term “about” means plus or minus 10% of the numerical value of the number with which it is being used. For example, “about 50%” means in the range of 45-55% and also includes exactly 50%. Where any value is described herein as modified by the term “about,” the exact value is also disclosed.

As used herein, the term “M” or “molarity” is used to refer to the concentration of a particular component in a solution, wherein M represents the moles of component per liter of solution.

As used herein, the term “contact lens” refers to any lens that is meant to be worn directly on the eyeball of a wearer. A contact lens may include a lens for vision correction, drug delivery, health monitoring, or other uses alone or in combination, according to the needs of the wearer of the contact lens.

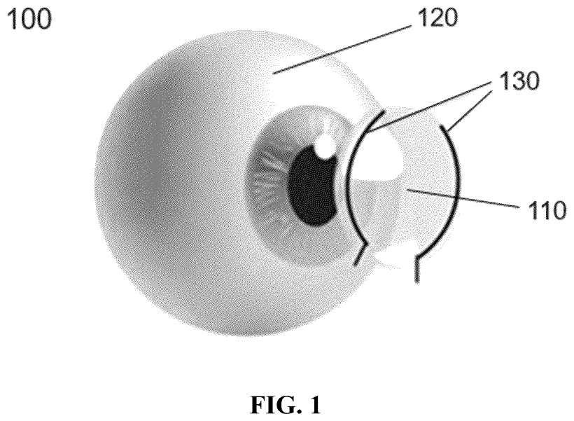

FIG. 1 is an illustrative diagram of a contact lens 100, according to embodiments of the present disclosure. The contact lens may include a lens 110 including a hydrogel configured to be worn on an eyeball 120 and an electronic component 130 fully encapsulated within the lens.

The lens 110 may include a hydrogel. The hydrogel may include polyvinyl alcohol (PVA), (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl]propyl methacrylate, silicone, polyethylene glycol, nanocellulose, or combinations thereof. The hydrogel may further include water.

The electronic component 130 fully encapsulated within the lens may include at least one of a sensor, a battery, a supercapacitor, an electronically controlled microfluidic channel, or combinations thereof. For example, in embodiments, the electronic component may include a battery and a supercapacitor. In embodiments, the electronic component may include a plurality of electronically controlled microfluidic channels, such as microfluidic channels controlled and/or powered by a micro-battery and any associated circuitry. For example, in embodiments, the electronic component may include a microfluidic channel which allows the electronic control of fluid flow. In embodiments, the electronic component may include a sensor and a supercapacitor. Any combinations of one or more of a sensor, a battery, a supercapacitor, an electronically controlled microfluidic channel, or combinations thereof are contemplated as possibilities for the electronic component.

In embodiments, the electronic component 130 may include carbon nanotubes, carbon allotropes, cellulose, or combinations thereof. The carbon nanotubes may include single-walled carbon nanotubes, double-walled carbon nanotubes, multi-walled carbon nanotubes, or combinations thereof. Carbon allotropes may include graphite, graphene, carbon black, carbon fibers, or other carbon structures known to those skilled in the art. The cellulose may include cellulose of from different sources, including but not limited to TEMPO-oxidized cellulose, bacterial cellulose, cellulose nanofibers, cellulose nanocrystals, and nanofibrillated cellulose. In embodiments, the carbon nanotubes and cellulose are combined in a mixture that includes about 2 wt. % to about 20 wt. % cellulose, for example, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 21 wt. %, about 22 wt. %, about 23 wt. %, about 24 wt. %, about 25 wt. %, or any value contained within a range formed by any two of the preceding values.

The electronic component 130 may be positioned on a substrate which is then fully encapsulated within the lens, or the electronic component may be fabricated on a substrate and then removed from the substrate prior to being fully encapsulated within the lens. In embodiments, the substrate may include a metal, a metal alloy or metal oxide, a polymeric material, a ceramic, a glass, a glass-laminated polymer, a composite, or combinations thereof. In embodiments, the substrate is selected to be compatible with the contact lens material. In embodiments, the substrate may have a predetermined shape, such as a round shape, a rectangular shape, a U-shape, a perforated square shape, a tube shape, a mesh shape, an interdigitated shape, or an I-shape. The carbon nanotube sheets produced by the method of the present disclosure may have a shape that is the same as the shape of the substrate. In embodiments, there may be multiple substrates which may have the same shapes or be of different shapes. In embodiments, the surface of the substrate may include a patterned or textured surface (e.g., a hammered, slotted, and/or perforated surface) or a non-patterned surface. In embodiments, the surface of the substrate may include a microscopic, patterned surface (e.g., a micro-pyramid structured surface, a micro-pillar structured surface) or a microscopic non-patterned surface (e.g., a smooth and/or polished surface).

According to embodiments of the present disclosure, the electronic component 130 may have a thickness of about 0.1 μm to about 40 μm. For example, the electronic component may have a thickness of about 0.1 μm, about 1 μm, 10 μm, about 15 μm, about 20 μm, about 25 μm, about 30 μm, about 35 μm, about 40 μm, or any value contained within a range formed by any two of the preceding values. In embodiments, the electronic component 130 may have a length of about 1.5 cm to about 2.0 cm, for example about 1.5 cm, about 1.6 cm, about 1.7 cm, about 1.8 cm, about 1.9 cm, about 2.0 cm, or any value contained within a range formed by any two of the preceding values, and a width of about 0.005 cm to about 2.0 cm, for example about 0.005 cm, about 0.01 cm, about 0.1 cm, about 1 cm, about 1.5 cm, about 1.6 cm, about 1.7 cm, about 1.8 cm, about 1.9 cm, about 2.0 cm, or any value contained within a range formed by any two of the preceding values.

The electronic component 130 may be fully encapsulated within the lens 110 such that no part of the electronic component is in contact with the eyeball 120. Preventing contact between the electronic component and the eyeball is important for ensuring biocompatibility and preventing harmful components from reaching the eyeball. In embodiments, the contact lens 100 is capable of utilizing naturally occurring fluids in the eye such as tears as an electrolyte, removing the need to use traditional electrolytes which may be toxic to the user.

According to embodiments of the present disclosure, the contact lens 100 may have a thickness of about 0.05 mm to about 0.5 mm. For example, the contact lens may have a thickness of about 0.05 mm, about 0.1 mm, about 0.15 mm, about 0.2 mm, about 0.25 mm, about 0.3 mm, about 0.35 mm, about 0.4 mm, about 0.45 mm, about 0.5 mm, or any value contained within a range formed by any two of the preceding values. The thickness of the contact lens 100 may vary depending on the thickness of the electronic component and the needs of a user of the contact lens. The thickness of the contact lens 100 should not exceed about 0.5 mm or 500 μm to ensure comfort for the user.

According to embodiments of the present disclosure, the contact lens 100 may be reusable. For example, the contact lens 100 may be worn by a user multiple times, consecutively or non-consecutively. In embodiments, the contact lens 100 may be worn about 2 times to about 14 times, for example, about 2 times, about 3 times, about 4 times, about 5 times, about 6 times, about 7 times, about 8 times, about 9 times, about 10 times, about 11 times, about 12 times, about 13 times, or about 14 times. Each time the contact lens is worn may include a wear time of about 1 hour to about 24 hours, for example about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or any value contained within a range formed by any two of the preceding values.

In embodiments, the contact lens 100 further includes a pharmaceutical which has been impregnated into the lens. The pharmaceutical may be introduced into the contact lens 100 by soaking the contact lens in a solution of the pharmaceutical such that the contact lens is impregnated with the pharmaceutical, or the pharmaceutical may be encapsulated within the lens. In embodiments, the pharmaceutical is released upon contact of the contact lens 100 with the eyeball 120. In embodiments, release of the pharmaceutical may be initiated by the electronic component. In embodiments, the contact lens 100 may be reimpregnated with the pharmaceutical after an initial use and release of the pharmaceutical. The composition of the pharmaceutical is not particularly limited. In embodiments, the pharmaceutical includes an antihistamine, an ophthalmic drug, or combinations thereof.

In embodiments, the contact lens 100 may be used in combination with a monitoring device, such as a sensor. In embodiments, the monitoring device measures one or more health parameters of the user and releases a pharmaceutical from an electronically controlled microfluidic channel within the contact lens 100 as needed, as determined by the monitoring device. As disclosed herein, the electronically controlled microfluidic channel may include a microfluidic channel powered by a battery which allows the electronic control of fluid flow. In embodiments, the electronic component 130 may be in communication with the monitoring device to monitor one or more health parameters of a user of the contact lens 100. The monitoring device may initiate the release of the pharmaceutical based on a set time interval or based on the one or more health parameters that are measured. For example, in embodiments, the monitoring device may initiate release of the pharmaceutical when a health parameter is above or below a particular value, without wishing to be bound by theory.

In embodiments, the contact lens may have a transparency of at least about 70%, such as about 70%, about 75%, about 80%, about 85%, about 90%, about 95%, about 100%, or any value contained within a range formed by any two of the preceding values.

In embodiments, the contact lens may have a water content of about 85 wt. % to about 99 wt. %, such as about 85 wt. %, about 86 wt. %, about 87 wt. %, about 88 wt. %, about 89 wt. %, about 90 wt. %, about 91 wt. %, about 92 wt. %, about 93 wt. %, about 94 wt. %, about 95 wt. %, about 96 wt. %, about 97 wt. %, about 98 wt. %, about 99 wt. %, or any value contained within a range formed by any two of the preceding values. The crosslinking time for the hydrogel may affect the water content of the contact lens, without wishing to be bound by theory.

In embodiments, the contact lens has an oxygen permeability of about 55 Barrer to about 85 Barrer, such as about 55 Barrer, about 60 Barrer, about 65 Barrer, about 70 Barrer, about 75 Barrer, about 80 Barrer, about 85 Barrer, or any value contained within a range formed by any two of the preceding values. about 99 wt. %, or any value contained within a range formed by any two of the preceding values. The crosslinking time for the hydrogel may affect the oxygen permeability of the contact lens, without wishing to be bound by theory.

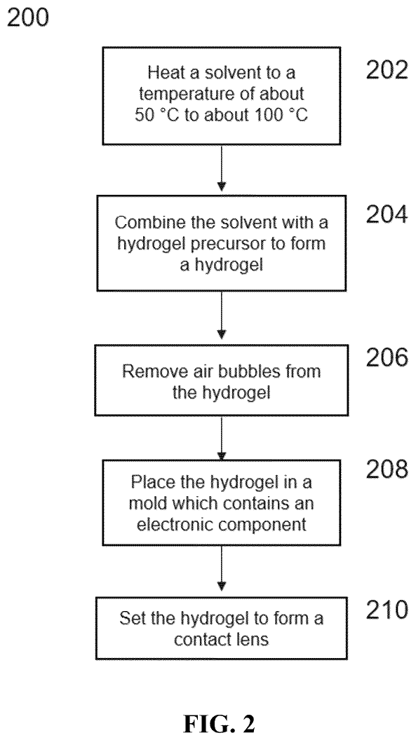

FIG. 2 is a flow chart of a method of making a contact lens, according to embodiments of the present disclosure. The method 200 may include steps of heating a solvent to a temperature of about 50° C. to about 100° C. 202, combining the solvent with a hydrogel precursor to form a hydrogel 204, removing air bubbles from the hydrogel 206, placing the hydrogel in a mold which contains an electronic component 208, and setting the hydrogel to form a contact lens 210.

The step of heating the solvent to a temperature of about 50° C. to about 100° C. 202 may include heating a solvent to a temperature of about 50° C., about 55° C., about 60° C., about 65° C., about 70° C., about 75° C., about 80° C., about 85° C., about 90° C., about 95° C., about 100° C., or any value contained within a range formed by any two of the preceding values. The temperature may be held constant, or it may be changed, such as heating first to about 50° C. and subsequently to about 70° C., heating first to about 60° C. and subsequently to about 80° C., heating first to about 70° C. and subsequently to about 90° C., heating first to about 80° C. and subsequently to about 100° C., or any value contained within a range formed by any two of the preceding values.

The method 200 may include a step of combining the solvent with a hydrogel precursor to form a hydrogel 204, according to embodiments of the present disclosure. The hydrogel precursor may include polyvinyl alcohol, (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl] propyl methacrylate, silicone, polyethylene glycol, nanocellulose, or combinations thereof. In embodiments, the hydrogel precursor may have an average molecular weight of about 85,000 g/mol to about 125,000 g/mol. Without wishing to be bound by theory, it is contemplated that the molecular weight of the hydrogel precursor may have an effect on the density and thus flexibility of the resulting lens, and thus a skilled artisan may adjust the molecular weight of the hydrogel precursor depending on the desired properties of the lens.

The hydrogel precursor may be added to the solvent in an amount of about 0.5 M to about 1.5 M relative to the solvent. In embodiments, the solvent includes water, dimethylsulfoxide, chloroform, dichloromethane, methanol, ethanol, or combinations thereof. For example, in embodiments, the solvent may include water and dimethylsulfoxide in a ratio of about 50:50 to about 10:90 water to dimethylsulfoxide, such as about 50:50, about 40:60, about 30:70, about 20:80, about 10:90, or any value contained within a range formed by any two of the preceding values. The step of combining the solvent with the hydrogel precursor may include adding the hydrogel precursor to the solvent in one portion or in multiple portions. In embodiments, combining the solvent with the hydrogel precursor may include stirring, sonicating, or combinations thereof. It is contemplated that the amount of DMSO included in the solvent is related to the pore size and crystallinity ratios of the resulting hydrogel. Without wishing to be bound by theory, the addition of DMSO to the solvent system may minimize volume expansion during freezing, relevant to water alone, leading to smaller pore sizes and higher crystallinity ratios with fewer freeze/thaw cycles required.

During the step of combining 204, the solvent and hydrogel precursor may be heated to a temperature of about 25° C. to about 100° C. In embodiments, the solvent and hydrogel precursor are not heated and the step of combining 204 is conducted at room temperature. The temperature may be maintained from the step of heating the solvent 202, or the temperature may be first cooled to room temperature and then adjusted to a temperature of about 25° C. to about 100° C. For example, in embodiments, the step of combining the solvent with the hydrogel precursor to form a hydrogel 204 may include heating to or maintaining a temperature of about 25° C. to about 100° C., such as about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., or any value contained within a range formed by any two of the preceding values.

The method 200 may include a step of removing air bubbles from the hydrogel 206. Removing air bubbles from the hydrogel 206 may include placing the hydrogel under vacuum, sonicating the hydrogel, or a combination thereof. In embodiments, the step of removing air bubbles from the hydrogel 206 is conducted for about 1 hour, about 2 hours, about 3 hours, about 4 hours, or any value contained within a range formed by any two of the preceding values. In embodiments, other methods of removing air bubbles from the hydrogel may be utilized. The method of removing air bubbles from the hydrogel is not particularly limited.

The method 200 may include a step of placing the hydrogel in a mold which contains an electronic component 208. In embodiments, the mold constructed via 3D printing and cellular lithography. The material of the mold is not particularly limited, and in embodiments, the mold is formed from silicon or Teflon. In embodiments, the electronic component may include comprises at least one of a sensor, a battery, a supercapacitor, an electronically controlled microfluidic channel, or combinations thereof. For example, in embodiments, the electronic component may include a battery and a supercapacitor. In embodiments, the electronic component may include a plurality of electronically controlled microfluidic channels. In embodiments, the electronic component may include a sensor and a supercapacitor. Any combinations of one or more of a sensor, a battery, a supercapacitor, an electronically controlled microfluidic channel, or combinations thereof are contemplated as possibilities for the electronic component. In embodiments, the electronic component includes carbon nanotubes and cellulose.

In embodiments, the mold includes a well to produce one contact lens. In embodiments, the mold includes a plurality of molds such that a plurality of contact lenses may be formed simultaneously. For example, the mold may include two wells to form two contact lenses, three wells to form three contact lenses, four wells to form four contact lenses, ten wells to form ten contact lenses, twenty wells to form twenty contact lenses, thirty wells to form thirty contact lenses, and so on. The number of wells in the mold and thus the number of contact lenses that may be formed at once is not particularly limited. In embodiments, the method includes using SLA 3D printed molds with the electrode shapes engraved therein for maintaining the position of the electronic device components during the encapsulation.

The step of placing the hydrogel in a mold which contains an electronic component 208 may include adding about 0.1 mL to about 0.5 mL of hydrogel per contact lens to be made to the mold. For example, step 208 may include adding about 0.1 mL, about 0.2 mL, about 0.3 mL, about 0.4 mL, about 0.5 mL, or any value contained within a range formed by any two of the preceding values, of hydrogel to the mold per each contact lens to be formed. Step 208 of placing the hydrogel in a mold which contains an electronic component may be performed by hand or by an automated system. In embodiments, the mold contains only part of an electronic component, such as one of a pair of electrodes. In embodiments, the mold contains a first part of the electronic component, and a first portion of the hydrogel is added to the mold, and the first portion of the hydrogel is crosslinked by the methods described herein such that the first part of the electronic component is fully encapsulated within the hydrogel. In embodiments, a second part of the electronic component may be added to the same mold, followed by subsequently adding a second portion of the hydrogel to the mold and crosslinking the second portion of the hydrogel, such that the resulting contact lens may be layered. It is contemplated that the hydrogel may be added to the mold in multiple portions, such that there may be a first portion, a second portion, a third portion, and so forth. Similar, the electronic component may, in embodiments, be divided into multiple parts such that there is a first part, a second part, a third part, and so forth, wherein each part of the electronic component may be contained within the mold prior to adding any hydrogel or may be adding after an addition of one or more portions of the hydrogel.

The method 200 may include a step of setting the hydrogel to form a contact lens 210. In embodiments, setting the hydrogel 210 includes photo-crosslinking. In embodiments, setting the hydrogel 210 may include physical crosslinking at a temperature of about −90° C. to about 25° C. For example, the temperature may be about −90° C., about −85° C., about −80° C., about −75° C., about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., or any value contained within a range formed by any two of the preceding values. The temperature may be maintained, or the temperature may be varied throughout the step of setting the hydrogel 210.

In embodiments, the step of setting the hydrogel 210 may include multiple cycles of freezing-thawing, such as 2 cycles to 10 cycles, for example 2 cycles, 3 cycles, 4 cycles, 5 cycles, 6 cycles, 7 cycles, 8 cycles, 9 cycles, or 10 cycles. Cycles of freezing-thawing may include varying the temperature as described above.

Setting the hydrogel 210 may include setting the hydrogel for a time of about 4 hours to about 24 hours, for example about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or any value contained within a range formed by any two of the preceding values.

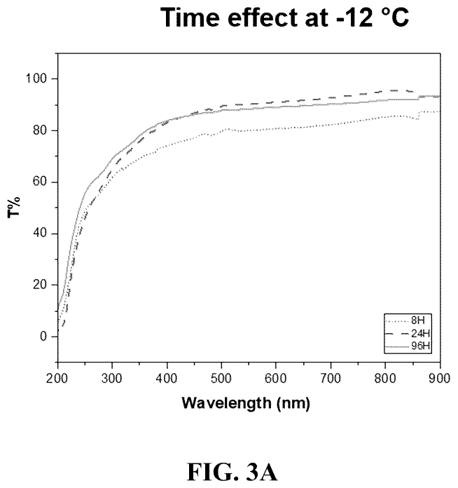

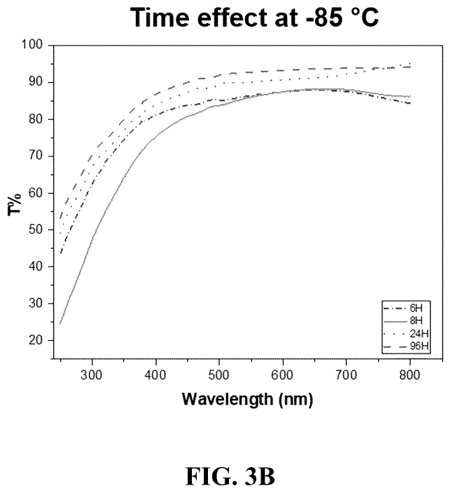

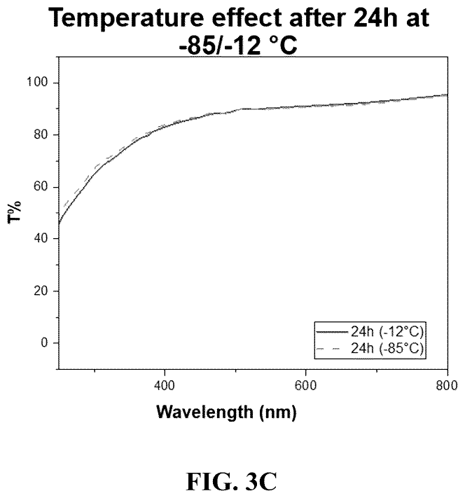

It is contemplated that the time and temperature of freezing may affect the transparency of the resulting lens, without wishing to be bound by theory. FIGS. 3A-3C are graphs of transparency of hydrogels formed with 5 wt. % PVA, over a time of 8 hours to 96 hours. FIG. 3A depicts the transparency of hydrogels containing 5 wt. % PVA at a freezing temperature of −12° C.; FIG. 3B depicts the transparency of hydrogels containing 5 wt. % PVA at a freezing temperature of −85° C.; and FIG. 3C compares the transparency of hydrogels containing 5 wt. % PVA at a freezing temperature of −85° C. and −12° C. over 24 hours.

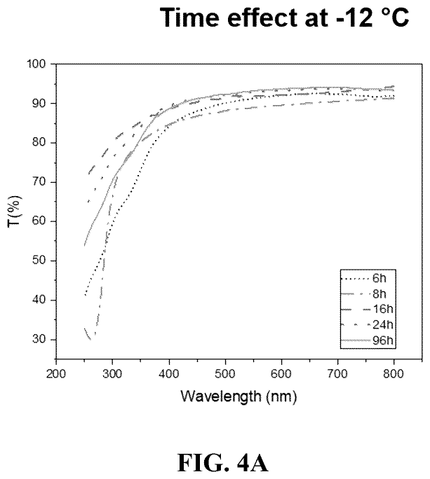

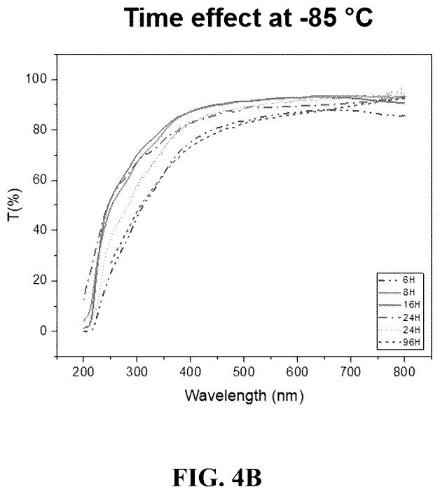

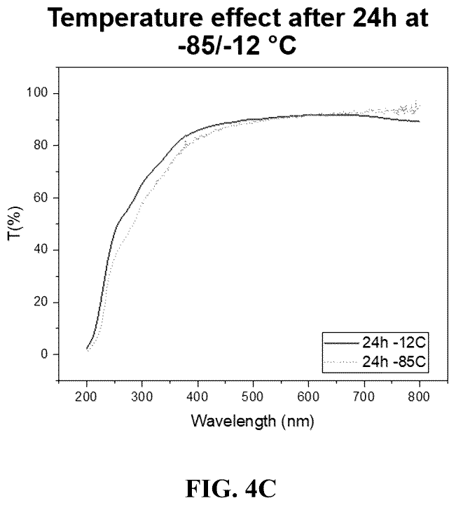

FIGS. 4A-4C are graphs of transparency of hydrogels formed with 7 wt. % PVA, over a time of 8 hours to 96 hours. FIG. 4A depicts the transparency of hydrogels containing 7 wt. % PVA at a freezing temperature of −12° C.; FIG. 4B depicts the transparency of hydrogels containing 7 wt. % PVA at a freezing temperature of −85° C.; and FIG. 4C compares the transparency of hydrogels containing 7 wt. % PVA at a freezing temperature of −85° C. and −12° C. over 24 hours.

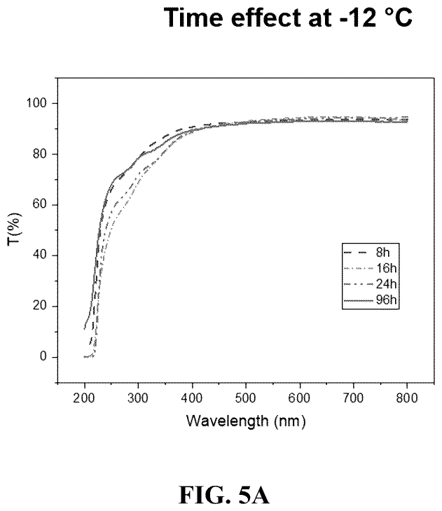

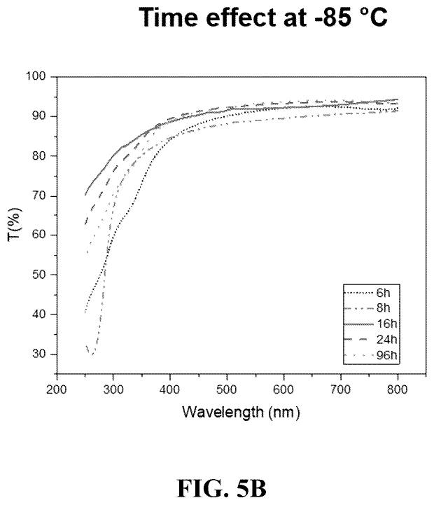

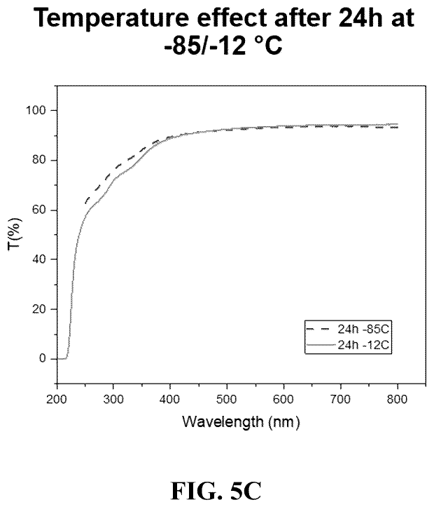

FIGS. 5A-5C are graphs of transparency of hydrogels formed with 10 wt. % PVA, over a time of 8 hours to 96 hours. FIG. 5A depicts the transparency of hydrogels containing 10 wt. % PVA at a freezing temperature of −12° C.; FIG. 5B depicts the transparency of hydrogels containing 10 wt. % PVA at a freezing temperature of −85° C.; and FIG. 5C compares the transparency of hydrogels containing 10 wt. % PVA at a freezing temperature of −85° C. and −12° C. over 24 hours.

Without wishing to be bound by theory, freezing at −12° C. and −85° C. for 24 hours yields hydrogels with similar transparency at 5 wt. %, 7 wt. %, and 10 wt. % PVA, which may allow the use of milder freezing temperature and lower energy expenditure.

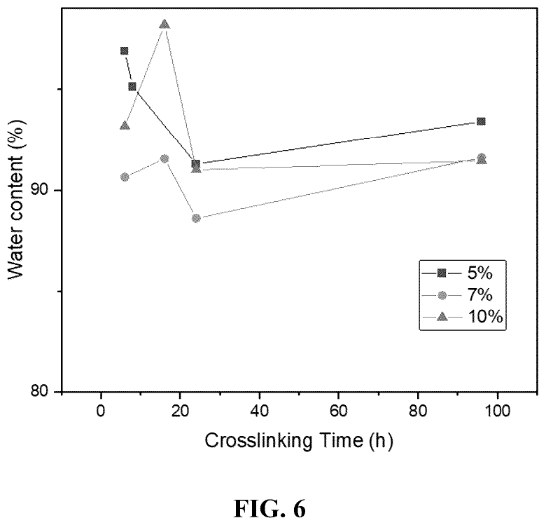

It is further contemplated that the crosslinking time for the hydrogel may affect the water content and oxygen permeability of the resulting contact lens. FIG. 6 is a graph of water content as a function of crosslinking time for hydrogels containing 5 wt. %, 7 wt. %, and 10 wt. % PVA, according to embodiments of the present disclosure. As shown, contact lenses containing 10 wt. % PVA and crosslinked for a time of about 18 hours reached the highest water content.

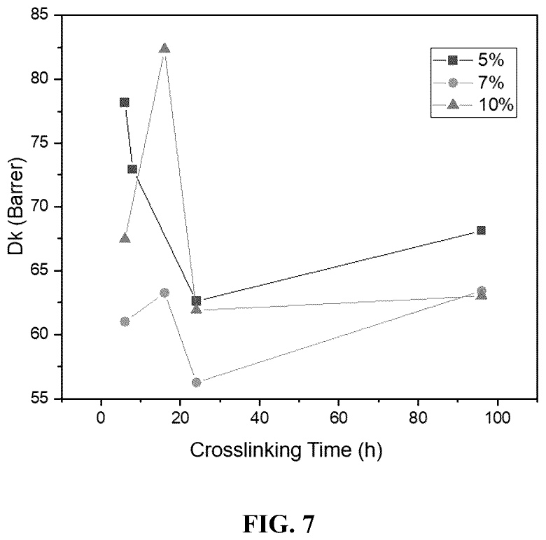

FIG. 7 is a graph of oxygen permeability as a function of crosslinking time for hydrogels containing 5 wt. %, 7 wt. %, and 10 wt. % PVA, according to embodiments of the present disclosure. As shown, contact lenses containing 10 wt. % PVA and crosslinked for a time of about 18 hours achieved the highest oxygen permeability.

In embodiments, setting the hydrogel 210 may include applying a force to the hydrogel during the freeze/thaw cycles. In embodiments, applying the force may include pressing, such as manual pressing or automated pressing.

The method 200 may include an optional step of impregnating the contact lens with a pharmaceutical such that the contact lens may be used for drug delivery. In embodiments, impregnating the contact lens with the pharmaceutical may include soaking the contact lens in a solution which includes the pharmaceutical, adding the pharmaceutical to the hydrogel, incorporating an electronically controlled microfluidic channel which includes the pharmaceutical into the contact lens, or combinations thereof. It is contemplated that the contact lens may be impregnated with the pharmaceutical multiple times, such that the contact lens may be reused for drug delivery. For example, the contact lens may be impregnated with the pharmaceutical a first time, used by a user of the contact lens, and then impregnated with the pharmaceutical a second time and used again.

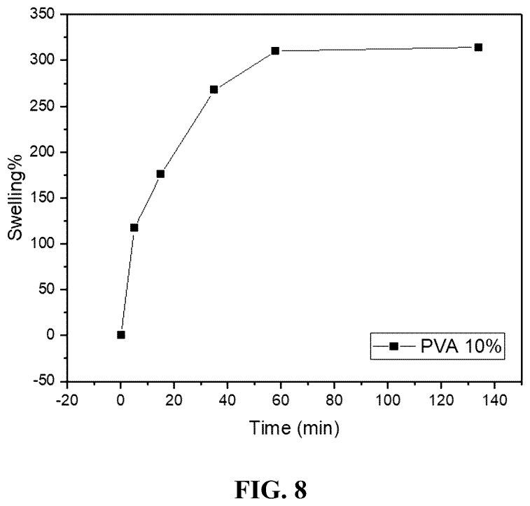

In embodiments, the contact lenses disclosed herein may exhibit a swelling percentage of greater than or equal to about 200%, such as about 200%, about 250%, about 300%, about 325%, or any value contained within a range formed by any two of the preceding values. FIG. 8 is a graph of percentage of swelling over time for lenses containing 10 wt. % PVA, according to embodiments of the present disclosure. Without wishing to be bound by theory, a swelling percentage of greater than or equal to about 200% may allow the lenses to maintain its comfort and flexibility while on the eye of the wearer.

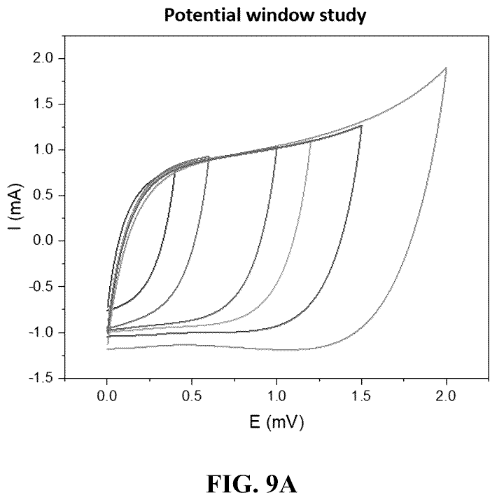

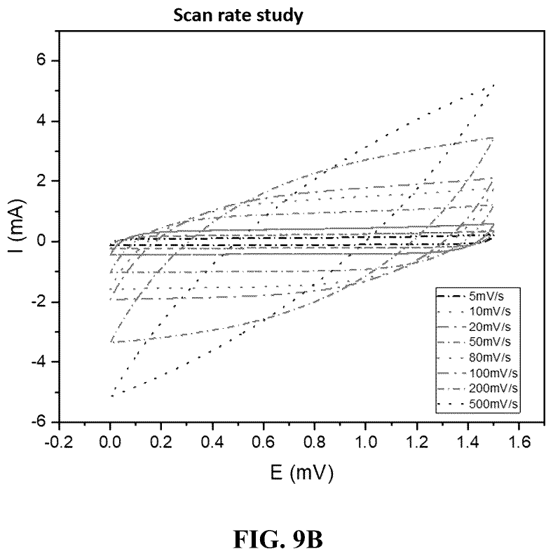

Electrochemical characterization of the contact lenses prepared by the methods disclosed herein was performed. FIG. 9A is a cyclic voltammogram evaluating the potential window of the hydrogels containing 10 wt. % PVA, according to embodiments of the present disclosure. FIG. 9B is a cyclic voltammogram of the hydrogels containing 10 wt. % PVA evaluating different scan rates. As shown in FIG. 9A, the presently disclosed contact lenses with an integrated electronic component achieve a high potential window up to 2 V. FIG. 9B shows that the presently disclosed contact lenses having 10 wt. % PVA with an integrated electronic component exhibit a capacitance of approximately 9 F/g in basic media, which suggests the presently discloses lenses exhibit electrochemical performance that is ideal for electric double layer capacitors, without wishing to be bound by theory.

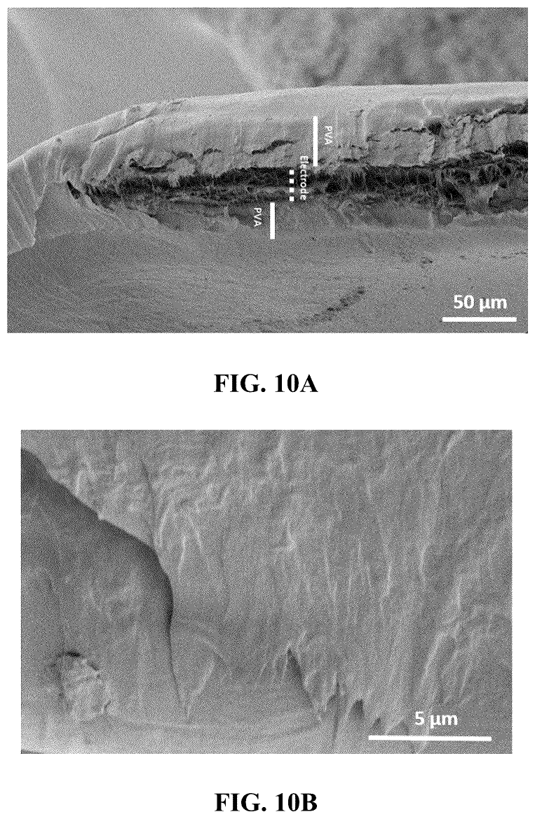

FIG. 10A and FIG. 10B show scanning electron microscopy of the contact lenses which were prepared according to an embodiment of the present disclosure. FIG. 10A shows the integration between the PVA component of the lens and the electronic component of the lens.

In embodiments, the contact lens of the present disclosure may be used for traditional vision correction, monitoring of health parameters, administration of pharmaceuticals, augmented reality (AR) or virtual reality (VR) integration, or combinations thereof. For example, the contact lens of the present disclosure may be used for oxygen monitoring of the eyeball for cancer detection, glucose monitoring, blood pressure monitoring, or the measurement of other health parameters. In embodiments, the contact lens of the present disclosure may be used for integration with an augmented or virtual reality system, and may provide the user with visual information including object identification, health and wellness parameters, entertainment experiences, or other features. The contact lens of the present disclosure may also provide traditional vision correction in place of or in addition to the above functions.

The embodiments disclosed herein may be combined in any manner to form new embodiments.

### EXAMPLES

### Example 1

A representative contact lens was prepared according to embodiments of the present disclosure. A solvent including dimethyl sulfoxide (DMSO) and water in an 80:20 ratio and in an amount of 20 mL was heated to about 60° C. Polyvinyl alcohol (PVA) having an average molecular weight of 85,000 to 124,000 g/mol (1 g) was added to the heated solvent in one portion and was stirred for about 1 hour as the temperature was increased to about 80° C. A transparent hydrogel was obtained and bath sonicated for about 2 hours to remove air bubbles. Concurrently, a female-male mold formed from one of silicon or Teflon and constructed via 3D printing and cellular lithography was provided. An electronic component including a supercapacitor including carbon nanotubes and cellulose electrodes was placed in the mold. Approximately 0.3 mL of the hydrogel was placed into the mold containing the electronic component. The hydrogel encapsulated the electronic component, and subsequently the hydrogel with encapsulated electronic component was physically crosslinked at a temperature of about 4° C. for a period of about 16-24 hours. During this stage of crosslinking, the hydrogel was subjected to 6 cycles of frost/defrost. After crosslinking, a flexible contact lens with a fully encapsulated electronic component was removed from the mold.

### Example 2

Approximately 16 mL of DMSO and 4 mL of water were heated to 55° C., and 1 g of PVA having an average molecular weight of 85,000 to 124,000 g/mol was added to the heated solvent mixture. The solution was homogenized for 2 hours, and then the temperature was increased to 65° C. for one hour. After dissolution of the PVA, the solution was placed under vacuum to remove air bubbles. The solution was reheated for 15-30 minutes, and then placed into the lens mold, followed by freezing at −25° C. for a minimum of 8 hours. The resulting lenses were washed with approximately 500 mL water for 24 to 48 hours to remove the DMSO.

This disclosure is not limited to the particular systems, devices and methods described, as these may vary. The terminology used in the description is for the purpose of describing the particular versions or embodiments only and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 compounds refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A contact lens, comprising:
a lens comprising a hydrogel configured to be worn on an eyeball, and
an electronic component fully encapsuled within the lens.

2. The contact lens of claim 1, wherein the hydrogel comprises polyvinyl alcohol, (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl]propyl methacrylate, silicone, polyethylene glycol, nanocellulose, or combinations thereof.

3. The contact lens of claim 1, wherein the electronic component comprises at least one of a sensor, a battery, a supercapacitor, an electronically controlled microfluidic channel, or combinations thereof.

4. The contact lens of claim 1, wherein the electronic component comprises carbon nanotubes, carbon allotropes, cellulose, or combinations thereof.

5. The contact lens of claim 1, wherein the contact lens has a thickness of 0.05 mm to 0.5 mm.

6. The contact lens of claim 1, wherein the contact lens has a thickness of 0.05 mm to 0.3 mm.

7. The contact lens of claim 1, wherein the contact lens is reusable.

8. The contact lens of claim 1, further comprising a pharmaceutical which has been impregnated into the contact lens.

9. The contact lens of claim 8, wherein the pharmaceutical comprises antihistamine, an ophthalmic drug, or combinations thereof.

10. The contact lens of claim 8, wherein the electronic component is in communication with a monitoring device to monitor one or more health parameters of a user of the contact lens.

11. The contact lens of claim 10, wherein the monitoring device initiates the release of the pharmaceutical according to the one or more health parameters.

12. A method of producing a contact lens, comprising:
heating a solvent to a temperature of 50° C. to 100° C.,
combining the solvent with a hydrogel precursor at a temperature of 25° C. to 100° C. to form a hydrogel,
placing an amount of the hydrogel into a mold comprising at least one well which contains an electronic component, and
setting the hydrogel to form a contact lens with the electronic component fully encapsulated therein.

13. The method of claim 12, wherein the solvent comprises water, dimethylsulfoxide, chloroform, dichloromethane, methanol, ethanol, or combinations thereof.

14. The method of claim 12, wherein the solvent comprises water and dimethylsulfoxide in a ratio of 50:50 to 10:90.

15. The method of claim 12, wherein the hydrogel precursor comprises polyvinyl alcohol, (hydroxyethyl)methacrylate, dimethyl methacrylate, N-vinyl pyrrolidone, ethylene glycol dimethacrylate, polydimethyl siloxane, hydroxyethyl methacrylate, 3-[tris(trimethylsiloxy)silyl] propyl methacrylate, silicone, polyethylene glycol, nanocellulose, or combinations thereof.

16. The method of claim 12, wherein combining the solvent with the hydrogel precursor comprises stirring, sonicating, or combinations thereof.

17. The method of claim 12, wherein the amount of hydrogel placed into the mold is 0.1 mL to 0.5 mL per contact lens.

18. The method of claim 12, wherein the electronic component comprises at least one of a sensor, a battery, a supercapacitor, an electronically controlled microfluidic channel, or combinations thereof.

19. The method of claim 12, wherein the mold comprises a plurality of wells containing an electronic component, such that a plurality of contact lenses with the electronic component fully encapsulated therein are formed simultaneously.

20. The method of claim 12, wherein setting the hydrogel comprises physical crosslinking at a temperature of −90° C. to 25° C. for a time of 4 hours to 24 hours.

21. The method of claim 12, further comprising impregnating the contact lens with a pharmaceutical such that the contact lens may be used for drug delivery.

22. The method of claim 21, wherein impregnating the contact lens with the pharmaceutical comprises soaking the contact lens in a solution comprising the pharmaceutical, adding the pharmaceutical to the hydrogel, incorporating an electronically controlled microfluidic channel comprising the pharmaceutical into the contact lens, or combinations thereof.
